(19) United States
(12) Patent Application Publication
(10) Pub. No.: US 2012/0196272 A1
(43) Pub. Date: Aug. 2, 2012

(54) Prediction of HCV Viral Kinetics in Interferon-Free Treatment

(75) Inventors: Tom Chu, Sunnyvale, CA (US); Uri A. Lopatin, San Francisco, CA (US); Nancy Shulman, Palo Alto, CA (US)

(73) Assignee: ROCHE MOLECULAR SYSTEMS, INC., Pleasanton, CA (US)

(21) Appl. No.: 13/194,248

(22) Filed: Jul. 29, 2011

Related U.S. Application Data
(60) Provisional application No. 61/370,903, filed on Aug. 5, 2010.

(51) Int. Cl. C12Q 1/70

(52) U.S. Cl. 435/5

(57) ABSTRACT

The present invention is based on the discovery of associations that exist between single nucleotide polymorphisms (SNPs) on chromosome 19 and virological outcomes in a diverse population of patients with hepatitis C virus (HCV) who received interferon-free treatment.

### CROSS REFERENCE TO RELATED INVENTION

This application claims the benefit of priority of U.S. Provisional Patent Application Ser. No. 61/370,903, filed Aug. 5, 2010, which is incorporated herein by reference in its entirety.

### FIELD OF THE INVENTION

The present invention relates to methods that useful for predicting the response of hepatitis C virus (HCV) infected patients to pharmacological treatment.

### BACKGROUND OF THE INVENTION

The standard of care for chronic hepatitis C is the combination of peginterferon plus ribavirin.¹ Overall sustained virological response (SVR) rates after treatment with the standard of care are approximately 50%,²⁻⁴ although it is difficult to predict whether an individual patient will achieve an SVR.

The probability of achieving an SVR varies with a collection of patient and viral factors. For example, younger patients, Caucasian and Asian patients, and individuals without advanced hepatic fibrosis are more likely to clear HCV infection after treatment.⁵⁻⁸ Similarly, patients infected with HCV genotypes 2 or 3, rather than genotype 1, and those with low baseline HCV RNA levels in serum have the best chance of a cure.^{2-4, 8}

More precise prediction of SVR is currently possible only after the start of treatment. Regardless of HCV genotype, individuals who clear HCV RNA after 4 or 12 weeks of treatment have a much better chance of achieving an SVR than those with persistent viremia.⁹ Rapid virological response (RVR, undetectable HCV RNA at week 4) is a strong predictor of SVR; conversely, failure to achieve an early virological response (EVR, greater than a two log decline in HCV RNA at week 12) is a strong predictor of nonresponse, independent of pretreatment characteristics.¹⁰

The ability to prospectively differentiate between potential responders and non-responders to the standard of care could have a great impact on the care of patients with chronic hepatitis C. Treatment decisions could be personalized based on the likelihood of patients to respond to the standard of care. For example patients with the lowest likelihood of achieving an SVR with the current standard of care might defer treatment until direct acting antiviral agents are available. Conversely, patients with a high likelihood of achieving an SVR might prefer to initiate therapy immediately with a treatment regimen that is a known entity.

In addition to host and viral factors, host genetic diversity also influences the response to treatment with the standard of care.¹¹ Recent evidence from genome-wide association studies suggests that single nucleotide polymorphisms (SNPs) in the promoter region of the IL-28b gene exert a strong influence on the probability of SVR in patients treated with peginterferon plus ribavirin.¹²⁻¹⁴ However, the impact of host IL-28b genotype on interferon-free regimens is unknown.

### SUMMARY OF THE INVENTION

The present invention is based on the discovery of an association between the SNP genotype at location rs12979860 and viral kinetics in patients treated with interferon-free regimens. In one embodiment, the invention provides for a method for predicting early viral load reduction of a human subject infected with HCV to interferon-free treatment that comprises at least one direct acting antiviral agent, comprising providing a sample from said human subject and identifying the nucleotide present at single nucleotide polymorphism rs12979860, wherein the presence of two C alleles at rs12979860 in said subject indicates a higher likelihood of early viral load reduction from said interferon-free treatment relative to a subject without two C alleles present at rs12979860.

In another embodiment, the invention provides for a method for predicting early viral load reduction of a human subject infected with HCV to interferon-free treatment that comprises at least one direct acting antiviral agent, comprising providing a sample from said human subject and identifying the nucleotide present at single nucleotide polymorphism rs12979860, wherein the presence of two T alleles or one T allele and one C allele at rs12979860 in said subject indicates a lower likelihood of early viral load reduction from said interferon-free treatment relative to a subject with two CC alleles present at rs12979860.

In another embodiment, the invention provides for a method of selecting a duration of interferon-free treatment that comprises at least one direct acting antiviral agent for achieving sustained virological response in a human subject infected with HCV, comprising providing a sample from said human subject and identifying the nucleotide present at single nucleotide polymorphism rs12979860, wherein the presence of two C alleles at rs12979860 in said subject indicates a shorter duration of said interferon-free treatment for achieving sustained virological response relative to a subject without two C alleles present at rs12979860.

In a further embodiment, the invention provides for a method for predicting response of a human subject infected with HCV to interferon-free treatment that comprises at least one direct acting antiviral agent, comprising providing a sample from said human subject and identifying the nucleotide present at single nucleotide polymorphism rs12979860, wherein the presence of two C alleles at rs12979860 in said subject indicates a higher likelihood of early virological response or sustained virological response achieved by said subject to said interferon-free treatment relative to a subject without two C alleles present at rs12979860.

In still a further embodiment, the invention provides for a method for predicting response of a human subject infected with HCV to interferon-free treatment that comprises at least one direct acting antiviral agent, comprising providing a sample from said human subject and identifying the nucleotide present at single nucleotide polymorphism rs12979860, wherein the presence of two T alleles or one T allele and one C allele at rs12979860 in said subject indicates a higher likelihood of no early virological response or sustained virological response achieved by said subject to said interferon-free treatment relative to a subject with two C alleles present at rs12979860.

### BRIEF DESCRIPTION OF THE DRAWINGS

FIG. 1. Distribution of viral load (IU/ml) decline by genotype (CC vs. non-CC), cohorts C, D, E, F, G at 14 days. Box represents 25-75^{th} percentile range of the distribution and central line represents the median. Dashed lines above and below the box represent 75-100^{th} percentile and 0-25^{th} percentile ranges of the distribution respectively. Blue dot represents distribution mean.

FIG. 2. Viral load (IU/ml) decline by genotype, cohorts F and G

### DETAILED DESCRIPTION OF THE INVENTION

Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “response” to treatment is a desirable response to the administration of an agent. “Interferon-free treatment” refers to treatment of patients without the use of exogenous interferon or pegylated interferon as defined herein below. Virological endpoints included “early virological response” (EVR), defined as ≧2-log drop in serum HCV RNA (“viral load”) from baseline to week 12 (by Cobas Amplicor HCV Monitor Test, v2.0, limit of quantitation 600 IU/mL), complete EVR (cEVR) defined as undetectable HCV RNA in serum (by Cobas Amplicor HCV Test v2.0, limit of detection 50 IU/mL) or and “sustained virological response” (SVR), defined as undetectable HCV RNA (<50 IU/mL) at the end of a 24-week untreated follow-up period.

The terms “sample” or “biological sample” refers to a sample of tissue or fluid isolated from an individual, including, but not limited to, for example, tissue biopsy, plasma, serum, whole blood, spinal fluid, lymph fluid, the external sections of the skin, respiratory, intestinal and genitourinary tracts, tears, saliva, milk, blood cells, tumors, organs. Also included are samples of in vitro cell culture constituents (including, but not limited to, conditioned medium resulting from the growth of cells in culture medium, putatively virally infected cells, recombinant cells, and cell components).

The terms “interferon” and “interferon-alpha” are used herein interchangeably and refer to the family of highly homologous species-specific proteins that inhibit viral replication and cellular proliferation and modulate immune response. Typical suitable interferons include, but are not limited to, recombinant interferon alpha-2b such as Intron® A interferon available from Schering Corporation, Kenilworth, N.J., recombinant interferon alpha-2a such as Roferon®-A interferon available from Hoffmann-La Roche, Nutley, N.J., recombinant interferon alpha-2C such as Berofor® alpha 2 interferon available from Boehringer Ingelheim Pharmaceutical, Inc., Ridgefield, Conn., interferon alpha-nl, a purified blend of natural alpha interferons such as Sumiferon® available from Sumitomo, Japan or as Wellferon® interferon alpha-nl (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alpha-n3 a mixture of natural alpha interferons made by Interferon Sciences and available from the Purdue Frederick Co., Norwalk, Conn., under the Alferon Tradename. The use of interferon alpha-2a or alpha-2b is preferred. Interferons can include pegylated interferons as defined below.

The terms “pegylated interferon”, “pegylated interferon alpha” and “peginterferon” are used herein interchangeably and means polyethylene glycol modified conjugates of interferon alpha, preferably interferon alfa-2a and alfa-2b. Typical suitable pegylated interferon alpha include, but are not limited to, Pegasys® and Peg-Intron®.

The term “ribavirin” refers to the compound, 1-((2R,3R,4S,5R)-3,4-Dihydroxy-5-hydroxymethyl-tetrahydro-furan-2-yl)-1H-[1,2,4]triazole-3-carboxylic acid amide which is a synthetic, non-interferon-inducing, broad spectrum antiviral nucleoside analog and available under the names, Virazole® and Copegus®.

“Direct acting antiviral agents” exert specific antiviral effects independent of immune function. Examples of direct acting antiviral agents for HCV include but are limited to protease inhibitors, polymerase inhibitors, NS5A inhibitors, IRES inhibitors and helicase inhibitors.

The terms “RG7128” and “R05024048” are used interchangeably and refer to the diisobutyl ester prodrug of the cytosine nucleoside analog b-D-2′-Deoxy-2′-fluoro-2′-C-methycytidine which is an inhibitor of the HCV NS5B RNA polymerase. Other HCV NS5B polymerase inhibitors include “ANA-598” from Anadys Pharmaceuticals, “ABT-333” from Abbott, “VX-222” from Vertex Pharmaceuticals, “BI-207127” from Boehringer Ingelheim, and “filibuvir” from Pfizer.

The terms “danoprevir”, “RG7227”, “R05190591” and “ITMN-191” are used interchangeably and refer to the macrocyclic peptidomimetic inhibitor of the HCV NS3/4A protease, 4-Fluoro-1,3-dihydro-isoindole-2-carboxylic acid (Z)-(1S,4R,6S,14S,18R)-14-tert-butoxycarbonylamino-4-cyclopropanesulfonylaminocarbonyl-2,15-dioxo-3,16-diaza-tricyclo[14.3.0.0*4,6*]nonadec-7-en-18-yl ester. Other HCV NS3 and NS3/4A protease inhibitors include, “boceprevir” or “SCH-503034”: (1R,5S)-N-[3-amino-1(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-(S)-carboxamide; and “telaprevir” or “VX-950”: (1S,3aR,6aS)-2-[(2S)-2-[[(2S)-cyclohexyl[(pyrazinylcarbonyeamino]acetyl]amino]-3,3-dimethylbutanoyl]-N—R1S)-1-[(cyclopropylamino)oxoacetyl]butyl]octahydrocyclopenta[c]pyrrole-1-carboxamide; “vaniprevir” or “ MK-7009”: (1R,21S,24S)-21-tert-Butyl-N-((1R,2R)-1-{[(cyclopropylsulfonyl)amino]carbonyl}-2-ethylcyclopropyl)-16,16-dimethyl-3,19,22-trioxo-2,18-dioxa-4,20,23-triazatetracyclo[21.2.1.1.0]heptacosa-6,8,10-triene-24-carboxamide; and “narlaprevir” or “SCH-900518”: (1R,2S,5S)-3-((S)-2-(3-(1-(tert-butylsulfonylmethyl)cyclohexyl)ureido)-3,3-dimethylbutanoyl)-N—((S)-1-(cyclopropylamino)-1,2-dioxoheptan-3-yl)-6,6-dimethyl-3-azabicyclo [3.1.0]hexane-2-carboxamide.

For patients with chronic hepatitis C (CHC) the current recommended first line treatment, referred as standard of care or SOC, is pegylated interferon alpha in combination with ribavirin for 48 weeks in patients carrying genotype 1 or 4 virus and for 24 weeks in patients carrying genotype 2 or 3 virus. Combined treatment with ribavirin was found to be more effective than interferon alpha monotherapy in patients who relapsed after one or more courses of interferon alpha therapy, as well as in previously untreated patients. However, ribavirin exhibits significant side effects including teratogenicity and carcinogenicity. Furthermore, ribavirin causes hemolytic anemia requiring dose reduction or discontinuation of ribavirin therapy in approximately 10 to 20% of patients, which may be related to the accumulation of ribavirin triphosphate in erythrocytes. Therefore, to reduce treatment cost and the incidence of adverse events, it is desirable to tailor the treatment to a shorter duration while not compromising efficacy. A shortened “duration of treatment” for genotype 1 patients with pegylated interferon alpha with ribovirin would be, for example, 24 weeks. A shortened duration of treatment for genotype 1 patients with a direct acting antiviral agent could be as short as 8 weeks, 12 weeks, or 16 weeks.

As used herein, the terms “allele” and “allelic variant” refer to alternative forms of a gene including introns, exons, intron/exon junctions and 3′ and/or 5′ untranslated regions that are associated with a gene or portions thereof. Generally, alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides.

As used herein, the term “polymorphism” refers to the coexistence of more than one form of a nucleic acid, including exons and introns, or portion (e.g., allelic variant) thereof. A portion of a gene of which there are at least two different forms, i.e., two different nucleotide sequences, is referred to as a polymorphic region of a gene. A polymorphic region can be a single nucleotide, i.e. “single nucleotide polymorphism” or “SNP”, the identity of which differs in different alleles. A polymorphic region can also be several nucleotides long.

Numerous methods for the detection of polymorphisms are known and may be used in conjunction with the present invention. Generally, these include the identification of one or more mutations in the underlying nucleic acid sequence either directly (e.g., in situ hybridization) or indirectly (identifying changes to a secondary molecule, e.g., protein sequence or protein binding).

One well-known method for detecting polymorphisms is allele specific hybridization using probes overlapping the mutation or polymorphic site and having about 5, 10, 20, 25, or 30 nucleotides around the mutation or polymorphic region. For use in a kit, e.g., several probes capable of hybridizing specifically to allelic variants, such as single nucleotide polymorphisms, are provided for the user or even attached to a solid phase support, e.g., a bead or chip.

The single nucleotide polymorphisms, “rs28416813”, “rs12979860”, and “rs810314” refer to SNPs identified by accession number in the database of SNPs (dbSNP, www.ncbi.nlm.nih.gov/SNP/) and are located on human chromosome 19 within the IL28b gene and its promoter region.

Utilization of baseline genetic predictors for interferon responsiveness allows tailoring of direct acting antiviral therapies, as well as standard of care with interferon. Patients who are defined as poorly interferon responsive, i.e. two T alleles in rs12979860 (or two C alleles of rs8103142 or 2 G alleles of rs2841683), may be poor candidates for small molecule therapeutics that rely on additive or synergistic effects of endogenous or exogenous interferon mediated pathways—particularly if these are provided as a single agent in addition to SOC. The concern would be that these patients would have an increased risk of developing drug resistant mutations as a result of effective monotherapy.

Conversely, patients with an interferon responsive phenotype, i.e. two C alleles in rs12979860 (or two T alleles of rs8103142 or two C alleles of rs2841683), would make superior candidates for shorter courses of therapy with SOC alone, or in combination with small molecules. Additional considerations that genetic predisposition to interferon responsiveness allow is “tuning” of combination direct acting antiviral agents. Patients predicted to have acceptable endogenous interferon responsiveness may be excellent candidates for drugs that target viral functions-such as protease inhibitors which also have an inhibitory role on endogenous interferon response—and poorer candidates for drugs that decrease the amounts of viral PAMP (pathogen associated molecular pattern, e.g. polymerase inhibitors) as these may serve to impair the patients capacity to facilitate their own cure via their endogenous interferon responsiveness. Similarly, patients predicted to have poor interferon responsiveness might be candidates for “quad” therapy as a first line of therapy (2 direct acting antiviral agents, added to peginterferon with ribavirin), as compared to a direct acting antiviral agent alone, or triple therapy (SOC with one DAA).

The results of analysis of the INFORM-1 trial (described in the Examples section), suggest that interferon responsive and poorly interferon responsive phenotypes display early differences to 2 combined direct acting antiviral agents for the treatment of CHC in the absence of SOC. As early responses are known to correlate with sustained virological response for CHC patients treated with SOC, markers of interferon responsiveness may potentially be used for guidance of interferon-free treatments. For example patient populations with two C alleles in rs12979860 (or two T alleles of rs8103142 or two C alleles of rs2841683), indicative of an interferon responsive phenotype, may achieve SVR with a shorter duration or reduced dosages of 2 or more combined direct acting antiviral agents without SOC compared with patient populations two T alleles in rs12979860 (or two C alleles of rs8103142 or 2 G alleles of rs2841683) indicative of a poorly interferon responsive phenotype. IL28B genotype in combination with clinical and other laboratory parameters may also be used to select the optimal treatment regimen for an individual patient among interferon-containing and interferon free treatments. By minimizing where possible the expected duration and intensity of treatment needed to achieve SVR, drug safety, in terms of adverse event rates, and efficacy, in terms of treatment compliance rates, may be improved.

### EXAMPLES

Methods

Study Design and DNA Sample Collections

Genotype analysis for IL28B polymorphisms was performed on patients with chronic hepatitis C (CHC) enrolled in the INFORM-1 trial. The aim of the INFORM-1 trial was to demonstrate that a combination of two experimental direct acting antivirals (DAAs) without pegylated-interferon or ribavirin, currently standard of care (SOC) for CHC, could be safely administered and provide significant antiviral activity without the emergence of resistance. The study medications consisted of RG7128 (also known as R05024048), the diisobutyl ester prodrug of the cytosine nucleoside analog β-D-2′-Deoxy-2′-fluoro-2′-C-methylcytidine which is an inhibitor of the HCV NS5B RNA polymerase and danoprevir (also known as RG7227, R05190591 and ITMN-191), the macrocyclic peptidomimetic inhibitor of the HCV NS3/4A protease. Both have potent in vitro and in vivo activity against HCV, and at the time of this study both compounds were in Phase I development.

INFORM-1 was a phase 1b, randomized, double-blind, placebo-controlled, dose-escalating trial. Eligible patients were males and females of non-child-bearing potential between 18 and 65 years, with chronic Genotype 1 HCV infection, without cirrhosis, and with a minimum baseline HCV RNA of 10⁵ IU/mL (Roche Taqman Assay). The study included SOC treatment-naïve, treatment failure (TF), and null responder patients. Treatment-naïve was defined as never having received an interferon-based treatment regimen for CHC; Treatment failure (non null) was defined as either relapsers (patients whose HCV RNA fell below the limit of detection while receiving SOC, but relapsed following discontinuation of therapy) or partial-responders (patients who had a HCV RNA reduction of at least 2 log₁₀ units after 12 weeks of treatment while on prior SOC but whose HCV RNA always remained detectable in blood). Null responders demonstrated less than 1 log₁₀ unit reduction in HCV RNA with one month and/or less than 2 log₁₀ reduction following 12 weeks of prior SOC therapy. Enrolled patients were randomized to either study treatment or placebo according to Table 1. Cohorts B-G received 13 days of experimental therapy. While all cohorts showed significant viral load reduction, cohorts C, D, F, and G received the highest doses of study drug with and had comparable exposures. These cohorts also demonstrated similar reductions in viral load.

TABLE 1
INFORM 1 Treatment schedule by cohort

| cohort | Prior experience with SOC | Danoprevir dose | RG7128 dose |
|---|---|---|---|
| B | naive | 100 mg q 8 h | 500 mg BID |
| C1 | naive | 100 mg q 8 h | 1000 mg BID |
| C2 | naive | 200 mg q 8 h | 500 mg BID |
| D | naive | 200 mg q 8 h | 1000 mg BID |
| E | Treatment failure (TF) | 600 mg BID | 1000 mg BID |
| F | null | 900 mg BID | 1000 mg BID |
| G | naive | 900 mg BID | 1000 mg BID |

Upon completion of study treatment, patients were permitted to initiate treatment with SOC at the discretion of the treating physician.

Plasma HCV RNA levels were measured with the COBAS TaqMan HCV assay, version 2, (Roche Molecular Systems), with a lower limit of quantification of 43 IU/mL and a lower limit of detection of 15 IU/mL HCV RNA levels were measured at the time of screening, at baseline, and on treatment at multiple times through the end of study drug administration and over 90 days of follow-up.

Additional details of the study design, inclusion and exclusion criteria and primary results of these trials are published elsewhere.

Blood samples for genetic analysis were collected from patients who consented to participate in genetic analyses were stored in the Roche Sample Repository. DNA was extracted at the Roche Sample Repository and normalized to 50 ng/μL.

Data Analysis

53 patients (37 treatment naive, 8 prior treatment failures (TFs) who were partial responders/relapsers to SOC, and 8 null responders to SOC) who received combination therapy at different doses for 14 days were studied. Naive patients were randomized to 4 treatment cohorts which differed in dosage and scheduling of treatment medications. Genotypes at the rs28416813, rs12979860, and rs8103142 loci were determined by direct sequencing of portions of the IL28B gene and upstream regions. Viral kinetic profiles during 13 days of interferon free therapy as well as after 4 and 12 weeks of subsequent SOC were compared by genotype. For analysis purposes, the lowest dosage cohort (B) was not considered.

Results

Overall frequencies of rs12979860 genotypes for all 53 INFORM 1 patients were CC—28.3%, CT—58.5%, and TT—13.2%. The CC genotype frequency was similar across the high dose cohorts (n=45) (Table 2). Genotype results for rs28416813 and rs8103142 were essentially identical to those for rs12979860.

TABLE 2
rs12979860 genotype distribution in
comparable dose cohorts C-G

| rs12979860 | naïve | | | TF | null | |
|---|---|---|---|---|---|---|
| Genotype | C | D | G | E | F | totals |

| | | | | | | | |
|---|---|---|---|---|---|---|---|
| CC | 6 | 2 | 3 | 1 | 0 | 12 | 27% |
| CT | 7 | 4 | 4 | 7 | 5 | 27 | 60% |
| TT | 1 | 2 | 0 | 0 | 3 | 6 | 13% |
| TOTAL | 14 | 8 | 7 | 8 | 8 | 45 | |

Differences (non-statistically significant owing to the small number of patients studied) in viral kinetics were observed over 13 days of IFN-free treatment when patients were stratified by rs12979860 genotype.

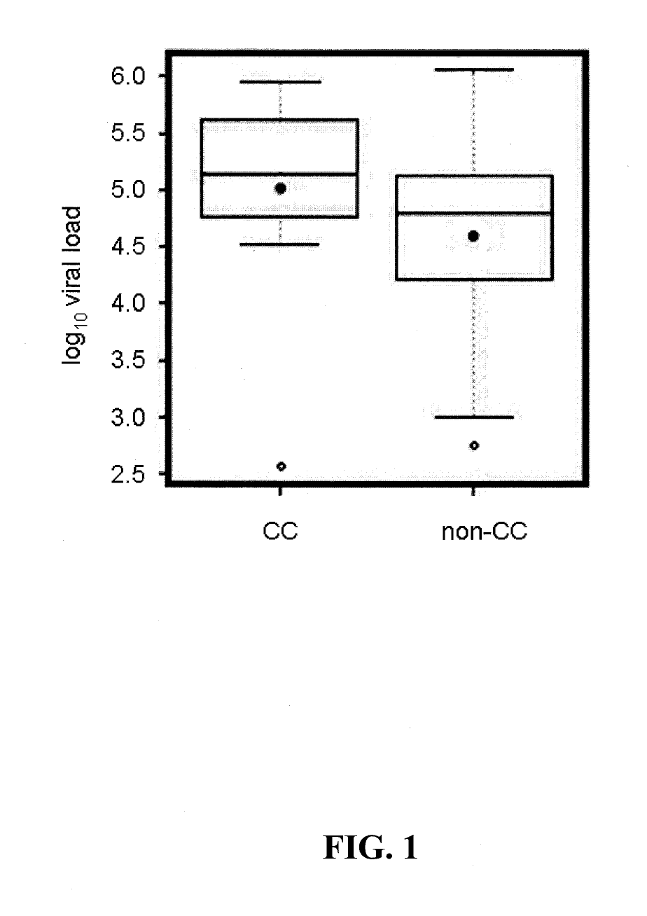

For patients receiving comparable doses of study drugs (FIG. 1, Table 3), those carrying the CC genotype of rs12979860 have a mean viral load reduction approximately 0.5 log 10 unit greater than other genotypes after 14 days of therapy and the distribution of responses is shifted compared to those with non-CC genotypes.

TABLE 3
Number of patients with undetectable HCV RNA
following DAA therapy

| Genotype | n | <15 IU/ml HCV RNA after 13 days DAAs |
|---|---|---|
| All Pts | 53 | |
| CC | 15 | 6 (40%) |
| non-CC | 38 | 10 (26%) |
| Comparable | 45 | |
| dose cohorts | | |
| CC | 12 | 6 (50%) |
| NON-CC | 33 | 9 (27%) |

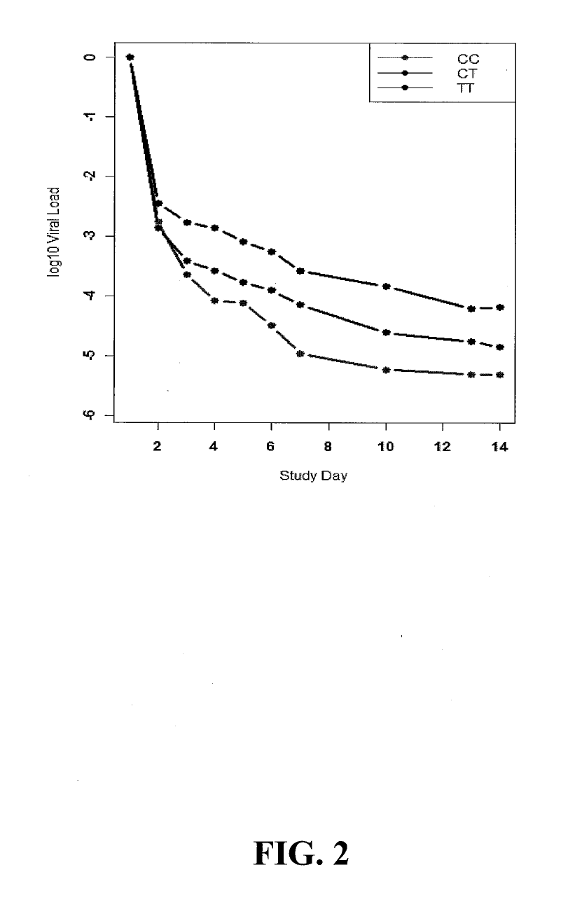

Differentiation of viral kinetics by genotype was most readily observed in patients who received the highest doses of study drug (FIG. 2).

These data suggest that IL28B genotype may influence early viral kinetics in patients receiving interferon-free treatments for hepatitis C, but appear to do so to a lesser extent than for interferon-containing treatments. This observation is consistent with the hypothesis that IL28B genotype information reflects endogenous interferon responsiveness. IL28B genotype information could inform selection of CHC treatment in terms of medications, dosage, or duration for both interferon free and interferon-containing regimens.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Ghany M G, Strader D B, Thomas D L, Seeff L B. Diagnosis, management, and treatment of hepatitis C: An update. Hepatology 2009; 49:1335-1374.
2. Manns M P, McHutchison J G, Gordon S C, Rustgi V K, Shiffman M, Reindollar R, Goodman Z D, Koury K, Ling M, Albrecht J K. Peginterferon alfa-2b plus ribavirin compared with interferon alfa-2b plus ribavirin for initial treatment of chronic hepatitis C: a randomised trial. Lancet 2001; 358:958-965.
3. Fried M W, Shiffman M L, Reddy K R, Smith C, Marinos G, Goncales F L, Jr., Haussinger D, Diago M, Carosi G, Dhumeaux D, Craxi A, Lin A, Hoffman J, Yu J. Peginterferon alfa-2a plus ribavirin for chronic hepatitis C virus infection. N Engl J Med 2002; 347:975-982.
4. Hadziyannis S J, Sette H, Jr., Morgan T R, Balan V, Diago M, Marcellin P, Ramadori G, Bodenheimer H, Jr., Bernstein D, Rizzetto M, Zeuzem S, Pockros P J, Lin A, Ackrill A M. Peginterferon-alpha2a and ribavirin combination therapy in chronic hepatitis C: a randomized study of treatment duration and ribavirin dose. Aim Intern Med 2004; 140:346-355.
5. Conjeevaram H S, Fried M W, Jeffers L J, Terrault N A, Wiley-Lucas T E, Afdhal N, Brown R S, Belle S H, Hoofnagle J H, Kleiner D E, Howell C D. Peginterferon and ribavirin treatment in African American and Caucasian American patients with hepatitis C genotype 1. Gastroenterology 2006; 131:470-477.
6. Dienstag J L, McHutchison J G. American Gastroenterological Association technical review on the management of hepatitis C. Gastroenterology 2006; 130:231-264.
7. Missiha S, Heathcote J, Arenovich T, Khan K. Impact of asian race on response to combination therapy with peginterferon alfa-2a and ribavirin in chronic hepatitis C. Am J Gastroenterol 2007; 102:2181-2188.
8. Reddy K R, Messinger D, Popescu M, Hadziyannis S J. Peginterferon alpha-2a (40 kDa) and ribavirin: comparable rates of sustained virological response in sub-sets of older and younger HCV genotype 1 patients. J Viral Hepat 2009; 16:724-731.
9. Ferenci P, Fried M W, Shiffman M L, Smith C I, Marinos G, Goncales F L, Jr., Haussinger D, Diago M, Carosi G, Dhumeaux D, Craxi A, Chaneac M, Reddy K R. Predicting sustained virological responses in chronic hepatitis C patients treated with peginterferon alfa-2a (40 KD)/ribavirin. J Hepatol 2005; 43:425-433.
10. Martinot-Peignoux M, Maylin S, Moucari R, Ripault M P, Boyer N, Cardoso A C, Giuily N, Castelnau C, Pouteau M, Stern C, Auperin A, Bedossa P, Asselah T, Marcellin P. Virological response at 4 weeks to predict outcome of hepatitis C treatment with pegylated interferon and ribavirin. Antivir Ther 2009; 14:501-511.
11. Asselah T, Bieche I, Sabbagh A, Bedossa P, Moreau R, Valla D, Vidaud M, Marcellin P. Gene expression and hepatitis C virus infection. Gut 2009; 58:846-858.
12. Ge D, Fellay J, Thompson A J, Simon J S, Shianna K V, Urban T J, Heinzen E L, Qiu P, Bertelsen A H, Muir A J, Sulkowski M, McHutchison J G, Goldstein D B. Genetic variation in IL28B predicts hepatitis C treatment-induced viral clearance. Nature 2009; 461:399-401.
13. Suppiah V, Moldovan M, Ahlenstiel G, Berg T, Weltman M, Abate M L, Bassendine M, Spengler U, Dore G J, Powell E, Riordan S, Sheridan D, Smedile A, Fragomeli V, Muller T, Bahlo M, Stewart G J, Booth D R, George J. IL28B is associated with response to chronic hepatitis C interferon-alpha and ribavirin therapy. Nat Genet 2009; 41:1100-1104.
14. Tanaka Y, Nishida N, Sugiyama M, Kurosaki M, Matsuura K, Sakamoto N, Nakagawa M, Korenaga M, Hino K, Hige S, Ito Y, Mita E, Tanaka E, Mochida S, Murawaki Y, Honda M, Sakai A, Hiasa Y, Nishiguchi S, Koike A, Sakaida I, Imamura M, Ito K, Yano K, Masaki N, Sugauchi F, Izumi N, Tokunaga K, Mizokami M. Genome-wide association of IL28B with response to pegylated interferon-alpha and ribavirin therapy for chronic hepatitis C. Nat Genet 2009; 41:1105-110

1. A method for predicting early viral load reduction of a human subject infected with HCV to interferon-free treatment that comprises at least one direct acting antiviral agent, comprising:
providing a sample from said human subject and identifying the nucleotide present at single nucleotide polymorphism rs12979860, wherein the presence of two C alleles at rs12979860 in said subject indicates a higher likelihood of early viral load reduction from said interferon-free treatment relative to a subject without two C alleles present at rs12979860.

2. The method of claim 1 wherein said direct acting antiviral agent is selected from a HCV protease inhibitor or a HCV polymerase inhibitor.

3. The method of claim 2 wherein said HCV protease inhibitor is selected from the group consisting of danoprevir, boceprevir, telaprevir, vaniprevir, and narlaprevir.

4. The method of claims 2 wherein said HCV polymerase inhibitor is selected from the group consisting of RG7128, ANA-598, ABT-333, VX-222, BI-207127, and filibuvir.

5. The method of claim 1 wherein said subject is infected with Genotype-1 HCV.

6. A method for predicting early viral load reduction of a human subject infected with HCV to interferon-free treatment that comprises at least one direct acting antiviral agent, comprising: providing a sample from said human subject and identifying the nucleotide present at single nucleotide polymorphism rs12979860, wherein the presence of two T alleles or one T allele and one C allele at rs12979860 in said subject indicates a lower likelihood of early viral load reduction from said interferon-free treatment relative to a subject with two CC alleles present at rs12979860.

7. The method of claim 6 wherein said direct acting antiviral agent is selected from a HCV protease inhibitor or a HCV polymerase inhibitor.

8. The method of claim 7 wherein said HCV protease inhibitor is selected from the group consisting of danoprevir, boceprevir, telaprevir, vaniprevir, and narlaprevir.

9. The method of claim 7 wherein said HCV polymerase inhibitor is selected from the group consisting of RG7128, ANA-598, ABT-333, VX-222, BI-207127, and filibuvir.

10. The method of claims 6 wherein said subject is infected with Genotype-1 HCV.

11. A method of selecting a duration of interferon-free treatment that comprises at least one direct acting antiviral agent for achieving sustained virological response in a human subject infected with HCV comprising: providing a sample from said human subject and identifying the nucleotide present at single nucleotide polymorphism rs12979860, wherein the presence of two C alleles at rs12979860 in said subject indicates a shorter duration of said interferon-free treatment for achieving sustained virological response relative to a subject without two C alleles present at rs12979860.

12. The method of claim 11 wherein said direct acting antiviral agent is selected from a HCV protease inhibitor or a HCV polymerase inhibitor.

13. The method of claim 12 wherein said HCV protease inhibitor is selected from the group consisting of danoprevir, boceprevir, telaprevir, vaniprevir, and narlaprevir.

14. The method of claim 12 wherein said HCV polymerase inhibitor is selected from the group consisting of RG7128, ANA-598, ABT-333, VX-222, BI-207127, and filibuvir.

15. The method of claims 11 wherein said subject is infected with Genotype-1 HCV.

16. A method for predicting response of a human subject infected with HCV to interferon-free treatment that comprises at least one direct acting antiviral agent, comprising providing a sample from said human subject and identifying the nucleotide present at single nucleotide polymorphism rs12979860, wherein the presence of two C alleles at rs12979860 in said subject indicates a higher likelihood of early virological response or sustained virological response achieved by said subject to said interferon-free treatment relative to a subject without two C alleles present at rs12979860.

17. The method of claim 16 wherein said direct acting antiviral agent is selected from a HCV protease inhibitor or a HCV polymerase inhibitor.

18. The method of claim 17 wherein said HCV protease inhibitor is selected from the group consisting of danoprevir, boceprevir, telaprevir, vaniprevir, and narlaprevir.

19. The method of claim 17 wherein said HCV polymerase inhibitor is selected from the group consisting of RG7128, ANA-598, ABT-333, VX-222, BI-207127, and filibuvir.

20. The method of claims 16 wherein said subject is infected with Genotype-1 HCV.

21. A method for predicting response of a human subject infected with HCV to interferon-free treatment that comprises at least one direct acting antiviral agent, comprising providing a sample from said human subject and identifying the nucleotide present at single nucleotide polymorphism rs12979860, wherein the presence of two T alleles or one T allele and one C allele at rs12979860 in said subject indicates a higher likelihood of no early virological response or sustained virological response achieved by said subject to said interferon-free treatment relative to a subject with two C alleles present at rs12979860.

22. The method of claim 21 wherein said direct acting antiviral agent is selected from a HCV protease inhibitor or a HCV polymerase inhibitor.

23. The method of claim 22 wherein said HCV protease inhibitor is selected from the group consisting of danoprevir, boceprevir, telaprevir, vanirprevir, and narlaprevir.

24. The method of claim 22 wherein said HCV polymerase inhibitor is selected from the group consisting of RG7128, ANA-598, ABT-333, VX-222, BI-207127, and filibuvir.

25. The method of claims 21 wherein said subject is infected with Genotype-1 HCV.
